(19) United States
(12) Patent Application Publication
(10) Pub. No.: US 2006/0052588 A1
(43) Pub. Date: Mar. 9, 2006

(54) Novel nitrosation polymer in organic synthesis

(76) Inventors: Didier Roche, Lyon (FR); Claude Lardy, Lyon (FR)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 10/529,640

(22) PCT Filed: Sep. 6, 2003
(86) PCT No.: PCT/EP03/09904

§ 371 Date: Mar. 31, 2005

(30) Foreign Application Priority Data
Oct. 1, 2002 (FR) 02/12136

(51) Int. Cl. C07C 69/86; C07C 69/84; C07C 69/76

(52) U.S. Cl. 534/586; 560/66; 525/261

(57) ABSTRACT

The present invention relates to a novel nitrosation polymer of the formula 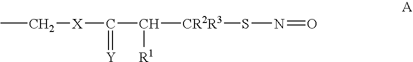
in which X, Y, R¹, R² and R³ are as defined in claim 1. The invention also relates to a process for the preparation of the said polymer.

The invention relates to a polymer that can be used in standard nitrosation reactions using sodium nitrite or alkyl nitrites as reagent. Examples that will be given include the nitrosation of secondary amines, the diazotisation of primary amines and the acetoxylation of a-amino acids.

The polymer according to the invention contains at least one thionitrite function (—S—N═O) grafted onto the polymer skeleton via covalent bonds. These reactive functions ensure the nitrosation reaction.

The advantages resulting from using the polymers of the invention in nitrosation, diazotisation and/or acetoxylation reactions are associated with the solid nature of the said polymers.

The polymers are removed from the reaction medium, after reaction, by simple filtration, thus allowing easy isolation of the reaction products.

According to the invention, the reactive polymers are readily regenerated after reaction to be reused in the same types of reaction. After regeneration, they have characteristics comparable with those of the freshly prepared polymers in terms of stability and reactivity.

Polymers containing —CH₂—N⁺(CH₃)₃.NO₂⁻ functions or isonitrile functions, which can be used in the preparation of azo dyes, have already been described in the art (cf. Green Chemistry, 2000, 43-45).

WO 99/67 296 and WO 98/05 689 also describe biodegradable polymers containing —S—NO or —NOₓ functions capable of releasing nitric oxide after implantation into the human body, the nitric oxide functioning as a platelet anti-aggregating agent.

More specifically, the polymers of the invention bear at least one function of the formula A: 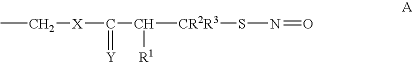
in which:
X represents O, S or NT, in which T represents H or a saturated aliphatic hydrocarbon-based group; Y represents O or S;
R¹ represents H; a saturated aliphatic hydrocarbon-based group, which is optionally substituted and/or optionally interrupted by one or more O or S atoms; a group —(O)ₚ—Ar, in which p represents the integer 0 or 1 and Ar represents an optionally substituted saturated and/or aromatic carbocyclic group or an optionally substituted saturated and/or aromatic heterocyclic group; a group Hyd-CO—O— or Hyd-CO—NH—, in which Hyd represents an optionally substituted saturated aliphatic hydrocarbon-based group; an optionally substituted aromatic group; or an optionally substituted aromatic heterocyclic group;
R² and R³ represent, independently of each other, a hydrogen atom; an optionally substituted saturated aliphatic hydrocarbon-based group; a saturated and/or aromatic carbocyclic group.

The expression “saturated aliphatic hydrocarbon-based group” more particularly means a linear or branched C₁-C₁₄, preferably C₁-C₈, for example C₁-C₆ and better still C₁-C₄ alkyl group.

Examples of alkyl groups are methyl, ethyl, propyl, isopropyl, butyl, isobutyl, t-butyl, pentyl, isopentyl, neopentyl, 2-methylbutyl, 1-ethylpropyl, hexyl, isohexyl, neohexyl, 1-methylpentyl, 3-methylpentyl, 1,1-dimethylbutyl, 1,3-dimethylbutyl, 2-ethylbutyl, 1-methyl-1-ethylpropyl, heptyl, 1-methylhexyl, 1-propylbutyl, 4,4-dimethylpentyl, octyl, 1-methylheptyl, 2-methylhexyl, 5,5-dimethylhexyl, nonyl, decyl, 1-methylnonyl, 3,7-dimethyloctyl and 7,7-dimethyloctyl.

The expression “optionally interrupted by one or more O or S atoms” means that in the aliphatic hydrocarbon-based chain constituting the hydrocarbon-based group, one or more carbon atoms may be replaced by one or more O or S atoms, it being understood that the resulting radical does not comprise two hetero atoms linked together. This expression is intended to include alkoxy and thioalkoxy groups, in which the alkyl portion is preferably as defined above.

The carbocyclic and heterocyclic radicals include monocyclic and polycyclic radicals; these radicals preferably denote monocyclic, bicyclic or tricyclic radicals. In the case of polycyclic radicals, it should be understood that these radicals consist of monocycles fused in pairs (for example ortho-fused or peri-fused), i.e. having at least two carbon atoms in common. Preferably, each monocycle is 3- to 8-membered and better still 5- to 7-membered.

The cycloalkyl groups are an example of saturated carbocyclic radicals and preferably contain from 3 to 18 and better still from 3 to 10 carbon atoms, such as cyclopropyl, cyclobutyl, cyclopentyl, cyclohexyl, cycloheptyl, cyclooctyl, adamantyl or norbornyl radicals.

The aromatic carbocyclic groups are, for example, C₆-C₁₈ aryl groups and especially phenyl, naphthyl, anthryl and phenanthryl.

The heterocyclic groups comprise hetero atoms generally chosen from O, S and N, optionally in oxidised form (in the case of S and N).

Preferably, each of the monocycles constituting the heterocycle comprises from 1 to 4 hetero atoms and better still from 1 to 3 hetero atoms.

The following are especially distinguished:
5- to 7-membered monocyclic heterocycles, for instance heteroaryls chosen from pyridine, furan, thiophene, pyrrole, le pyrazole, imidazole, thiazole, isoxazole, isothiazole, furazane, pyridazine, pyrimidine, pyrazine, thiazines, oxazole, pyrazole, oxadiazole, triazole and thiadiazole; and also the saturated derivatives thereof. Examples of 5- to 7-membered saturated heterocycles are especially tetrahydrofuran, dioxolane, imidazolidine, pyrazolidine, piperidine, dioxane, morpholine, dithiane, thiomorpholine, piperazine, trithiane, oxepine and azepine;
bicyclic heterocycles in which each monocycle is 5- to 7-membered, for instance heteroaryls chosen from indolizine, indole, isoindole, benzofurazane, benzothiophene, indazole, benzimidazole, benzothiazole, benzofurazane, benzothiofurazane, purine, quinoline, isoquinoline, cinnoline, phthalazine, quinazoline, quinoxaline, naphthyridines, pyrazolotriazine (such as pyrazolo-1,3,4-triazine), pyrazolopyrimidine and pteridine; and also the saturated derivatives thereof;
tricyclic heterocycles in which each monocycle is 5- to 7-membered, whether they are completely aromatic, for instance acridine, phenazine or carbazole, or not, such as saturated derivatives thereof, phenothiazine or phenoxazine.

It should be understood that the expression “saturated and/or aromatic cyclic (heterocyclic or carbocyclic) radical” means that the said radical may comprise a saturated portion and/or an aromatic portion.

Mention is made, for example, of the case of the following carbocyclic radicals: 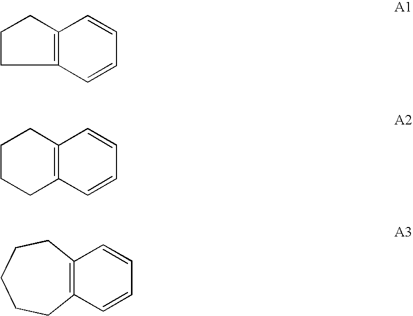
and also the case of the following heterocyclic radicals: 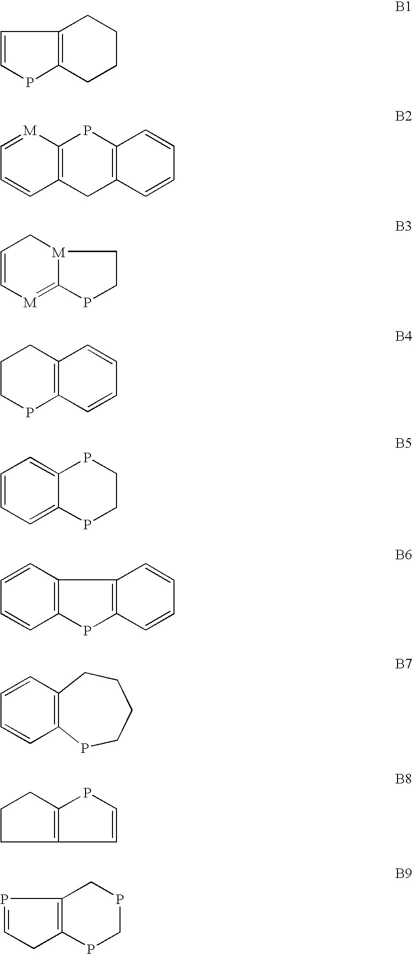
in which P represents O, S or SO₂ and M represents N or C. Preferably, in B1, P represents O or S; in B2, P represents SO₂ or 0 and M represent C or N; in B3, M represents N and P represents S; in B4, P represents O; in B5, P represents O; in B6, P represents O; in B7, P represents O; in B8, P represents S; in B9, P represents N.

If M or P represents N, this atom is preferably substituted by a hydrogen atom, alkyl or alkylcarbonyl.

The aliphatic hydrocarbon-based groups, the carbocyclic groups and the heterocyclic groups, which may be aromatic or saturated, are optionally substituted. The substituents may be of any nature according to the invention, provided that they do not interfere with the nitrosation, diazotisation or acetoxylation reaction.

Examples of substituents that may be envisaged include a halogen atom; cyano; hydroxyl; nitro; optionally halogenated (C₁-C₁₀)alkyl; optionally halogenated (C₁-C₁₀)alkoxy; (C₆-C₁₀)alkylthio optionally substituted by (C₆-C₁₀)-arylsulphonyl, in which aryl is optionally substituted by one or more radicals G; (C₆-C₁₀)aryloxy, in which aryl is optionally substituted by one or more radicals G; (C₆-C₁₀)arylthio, in which aryl is optionally substituted by one or more radicals G; (C₁-C₁₀)alkylsulphonyl; (C₆-C₁₀)arylsulphonyl, in which aryl is optionally substituted by one or more radicals G; 5- to 7-membered heteroaryl which comprises one or more hetero atoms chosen from O, N and S and is optionally substituted by one or more radicals G and/or by (C₁-C₁₀)alkoxycarbonyl; (C₁-C₁₀)-alkoxycarbonyl; (C₁-C₁₀)alkylcarbonylamino; di(C₁-C₁₀)alkylamino; (C₂-C₄)-alkylenedioxy; (C₃-C₅)alkylene, which is optionally substituted by oxo; (C₆-C₁₀)— aryl(C₁-C₁₀)alkyl, in which aryl is optionally substituted by one or more radicals G; optionally halogenated (C₆-C₁₀)aryl; (C₁-C₁₀)alkylcarbonyl, preferably (C₁-C₆)-alkylcarbonyl; (C₃-C₈)cycloalkyl(C₁-C₈)alkyl, in which cycloalkyl is itself substituted by (C₆-C₁₀)arylsulphonylamino, in which aryl is optionally halogenated; in which G is chosen from halogen; hydroxyl; optionally halogenated (C₁-C₁₄)-alkoxy, preferably optionally halogenated (C₁-C₁₀)alkoxy; optionally halogenated (C₁-C₁₄)alkyl, preferably optionally halogenated (C₁-C₁₀)alkyl; nitro; cyano; di-(C₁-C₁₄)alkylamino, preferably di(C₁-C₁₀)alkylamino; (C₆-C₁₀)aryl, which is optionally halogenated and/or optionally substituted by (C₁-C₁₄)alkyl.

According to the invention, the term “halogen atom” means a chlorine, bromine, fluorine or iodine atom.

The term “alkylene” means a linear or branched divalent hydrocarbon-based radical containing 1 to 6 carbon atoms, preferably 1 to 4 carbon atoms and better still 1 to 2 carbon atoms, derived from the removal of two hydrogen atoms on two different carbon atoms of a saturated hydrocarbon-based chain. The —CH₂— and —CH₂—CH₂ groups constitute alkylene radicals that are particularly preferred.

The polymers of the invention consist of a polymer skeleton to which is covalently attached at least one function A as defined above.

Examples of polymers include polymers with a skeleton of polysilicate, polyester, polyamide, polyurea, polythiourea, polyimide, polycarbonate, polyterephthalate, polysulphone, polystyrene or polyethylene glycol type, these last two polymers being preferred.

According to another embodiment of the invention, the polymer skeleton consists of a copolymer comprising two or more polymer chains chosen from polysilicate, polyester, polyamide, polyurea, polythiourea, polyimide, polycarbonate, polyterephthalate, polysulphone, polystyrene and polyethylene glycol. An example of such a copolymer is a polystyrene/polyethylene glycol copolymer.

However, the nature of the polymer skeleton is not critical according to the invention, provided that it is in solid form.

According to a more preferred embodiment of the invention, the polymer is in the form of particles, beads or solid spheres.

Thus, a person skilled in the art may select any solid polymer skeleton, preferably one that can be conditioned in the form of particles, beads or solid spheres.

An example of a polymer skeleton in particulate form that may be mentioned is functionalised silica.

An example of a polymer skeleton in the form of beads that may be mentioned is polystyrene copolymerised with 1-2% divinylbenzene.

However, the polymer skeleton may be in the form of a functionalised film, a lantern or a crown, or any other form known to those skilled in the art.

The term “polymer charge” denotes the number of moles of thionitrite functions per gram of polymer.

The charge is expressed in mmol/g and is preferably between 0.4 and 6 mmol/g and better still between 0.5 and 3.5 mmol/g, for example between 1 and 3 mmol/g.

A preferred subgroup of polymers is the group consisting of polymers for which X represents N.

Similarly, polymers that are preferred are those for which R² and R³ independently represent H, (C₁-C₈)alkyl; (C₆-C₁₀)aryl; and R¹ represents H; (C₁-C₈)alkyl; (C₆-C₁₀)aryl; (C₆-C₁₀)aryloxy; heteroaryl comprising one or more hetero atoms chosen from O, S and N and consisting of one or more 5- to 8-membered monocycles; heteroaryloxy in which heteroaryl is as defined above; (C₁-C₈)alkylcarbonyloxy; (C₁-C₈)alkylcarbonylamino; (C₆-C₁₀)arylcarbonyloxy; (C₆-C₁₀)arylcarbonylamino; heteroarylcarbonyloxy; or hetero-arylcarbonyl-amino; in which heteroaryl is as defined above.

Among this subgroup of polymers, the ones that are preferred are those for which R² and R³ independently represent (C₁-C₄)alkyl; and R¹ represents (C₁-C₄)-alkylcarbonylamino.

The function A is preferably
—CH₂—NH—CO—CH(N H—W)—C(CH₃)₂—S—N═O A1;
in which W represents alkanoyl (i.e. alkylcarbonyl), such as acetyl.

The polymers of the invention are readily prepared from corresponding polymers containing —CH₂—XH functions in which X is as defined above.

Suitable polymers containing —CH₂—XH functions are especially functionalised Merrifield polymers, such as those described in J. Am. Chem. Soc. 1963, 85, 2149.

The term “corresponding polymer” means the polymer having an identical polymer skeleton and containing —CH₂—XH functions instead of each function A.

More specifically, the process of the invention comprises the steps consisting in:
a) reacting a precursor polymer P bearing at least one —CH₂—XH function with the required amount of a reagent of the formula I: 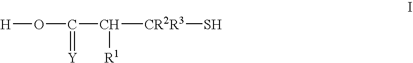
   in which the HO—C(═Y)— function is optionally in activated form and Y, R¹, R² and R³ are as defined above, such that each —CH₂—XH function of the polymer P reacts with a molecule of the reagent of the formula I, and then
b) treating the resulting polymer bearing at least one function B: 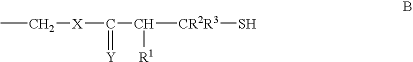
   with a nitrosating agent, so as to convert each of the functions B into functions C:

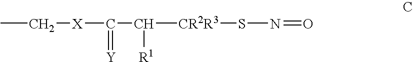

The precursor P contains an identical number of —CH₂—XH functions (i.e. an identical charge) to the number of functions A present in the polymer to be prepared.

If the function HO—C(═Y)— of the compound of the formula I is in activated form, the corresponding activated derivative has the formula IB 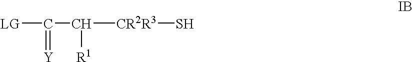
in which LG represents a leaving group and Y, R¹, R² and R³ are as defined above.

Examples of groups LG especially include a halogen atom (and more particularly a chlorine atom); an azide group; imidazolide; p-nitrophenoxy; 1-benzotriazole; N-succinimide; acyloxy (such as pivaloyloxy); (C₁-C₄ alkoxy)carbonyloxy; dialkyl- or dicycloalkyl-O-ureide.

Preferably, LG is the N-succinimide group of the formula:

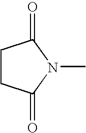

If the compounds of the formula I are used in unmodified form, without activation of the function HO—C(═Y)—, the reaction of the polymer with the compound of the formula I is performed at high temperature (between 40 and 200° C.) or at a lower temperature (between 10 and 150° C.) in the presence of a coupling agent, for instance a carbodiimide, optionally in the presence of an activating agent, for instance hydroxybenzotriazole or hydroxysuccinimide.

Representative coupling agents are dicycloalkyl- and dialkylcarbodiimides, carbodlimides that are soluble in an aqueous medium and especially dicyclohexylcarbodiimide, diisopropylcarbodiimide and (3-dimethylaminopropyl)-3-ethylcarbodiimide.

If the function HO—C(═Y)— is activated, the reaction is performed at a temperature that can range between 10 and 120° C., for example between 15 and 30° C.

The reaction is preferably performed in a polar solvent, such as a halogenated aliphatic or aromatic hydrocarbon (dichloromethane, chloroform or a chlorobenzene); a ketone, such as acetone; a nitrile, such as acetonitrile; an amide, such as acetamide, formamide or dimethylformamide; or an ether, such as tetrahydrofuran, diethyl ether, diisopropyl ether, dioxane or dimethoxyethane.

The solvent is preferably dimethylformamide.

Insofar as the polymer containing —CH₂—XH functions is preferably in solid form, it is to a suspension of this polymer in an inert solvent that a solution of compound I in an inert solvent is added. Preferably, the solvent for the suspension and the solvent for the solution of compound I are identical.

The preferred reaction conditions are those that envisage the use of equimolar amounts of substances reacting in inert solvents.

However, although stoichiometric amounts of the functions reacting together are sufficient, it is preferable to perform the process in the presence of an excess of the compound of the formula I. Thus, the molar ratio of the compound of the formula I to the functions —CH₂—XH preferably ranges between 1 and 10, for example between 1 and 6 and better still between 1 and 5.

If the process is performed in the presence of a coupling agent, this agent is generally used in stoichiometric amount relative to the amount of compound of the formula I used.

In step b), the nitrosation is performed by treating the polymer resulting from step a) using any nitrosating agent. Suitable examples of nitrosating agents include an alkali metal nitrite (such as sodium nitrite), an alkyl nitrite (preferably a C₁-C₆ alkyl nitrite), such as ethyl nitrite or tert-butyl nitrite, or NO⁺BF₄⁻.

The process is advantageously performed in the presence of sodium nitrite.

A person skilled in the art will readily establish the operating conditions.

If the nitrosating agent is an alkali metal nitrite, the process will usually be performed in a polar solvent, such as a mixture of ether, water and a carboxylic acid.

Acetic acid is preferred as carboxylic acid.

Cyclic ethers, such as dioxane and tetrahydrofuran are preferred as ether. As a variant, an ether, such as diethyl ether, diisopropyl ether or dimethoxyethane may also be used.

In this mixture, the volume ratio of the ether to water ranges between 20 and 5 and preferably between 12 and 8.

In this mixture, the volume ratio of acetic acid to water ranges between 1.5 and 5 and better still between 1.5 and 3.

The reaction of step b) is preferably performed between 15 and 35° C.

The reactive polymers of the invention can be used, for example, as reagents for the N-nitrosation of secondary amines, for the diazotisation of primary amines and for the acetoxylation of a-amino acids.

According to another of its aspects, the invention thus relates to a process for nitrosating secondary amines which consists in reacting a secondary amine with a polymer according to the invention so as to obtain the corresponding nitroso derivative.

Preferably, the nitrosation is performed in the presence of an excess of reactive functions of the formula A relative to the amount of secondary amine functions present.

According to one preferred embodiment of the invention, the molar ratio of the functions of the formula A of the polymer to the secondary amine functions ranges between 2 and 10 and preferably between 2 and 5.

Advantageously, the nitrosation reaction is performed at a temperature of between 15 and 35° C. and better still between 20 and 25° C.

The nitrosation reaction is preferably performed in a polar solvent, such as an aliphatic or aromatic halogenated hydrocarbon (such as dichloromethane, chloroform or chlorobenzene); an ether, such as tetrahydrofuran, dioxane, diethyl ether, diisopropyl ether or dimethoxyethane; a nitrile, such as acetonitrile; an amide, such as acetamide or dimethylformamide; or one of these solvents in deuterated form, i.e. in which one or more of the hydrogen atoms have been replaced by deuterium atoms. In a particularly advantageous manner, the solvent is CDCl₃ or chloroform.

The advantage of this process is the ease of monitoring the reaction progress by thin layer chromatography or LC-MS (mass spectrography coupled to liquid chromatography).

Another advantage is the ease with which the nitrosation reaction product is isolated, by simple filtration of the reaction medium and evaporation and/or removal of the solvents.

The nitrosation process of the invention applies more particularly to secondary amines chosen from:
diphenylamines optionally substituted by one or more substituents chosen from alkyl, alkoxy, cyano and hydroxyl;
the amines of the formula: 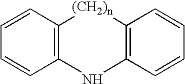
in which n is an integer equal to 0, 1, 2 or 3 and the phenyl nuclei are optionally independently substituted by alkyl, alkoxy, cyano or hydroxyl;
phenylalkylamines, which are optionally substituted by one or more substituents chosen from alkyl, alkoxy, cyano and hydroxyl;
benzopyrrolidines and benzopiperidines, which are optionally substituted by one or more substituents chosen from hydroxyl, alkyl, cyano and alkoxy;
benzylpyrrolidines, which are optionally substituted by one or more substituents chosen from hydroxyl, alkyl, cyano and alkoxy; and
benzylpiperidines, which are optionally substituted by one or more substituents chosen from hydroxyl, alkyl, cyano and alkoxy.

According to another aspect, the invention relates to a process for diazotising primary amines which consists in reacting a primary amine with a polymer according to the invention, so as to obtain the corresponding diazonium derivative. In this case also, the process is preferably performed in the presence of an excess of reactive functions of the formula A relative to the amount of primary amine functions.

Advantageously, the molar ratio of the functions of the formula A relative to the primary amine functions ranges between 2 and 10 and preferably between 2 and 5.

Advantageously, the reaction temperature ranges between −10 and 35° C. and better still between 20 and 25° C.

The solvents that are suitable for this reaction are those mentioned above for the nitrosation reaction. The process is preferably performed in dichloromethane or CD₂Cl₂.

It is particularly desirable to add to the reaction medium a C₁-C₄ carboxylic acid, such as acetic acid, in an at least stoichiometric amount relative to the amount of primary amine present.

The advantages of this reaction are the same as those mentioned above: easy monitoring of the reaction progress and easy isolation of the reaction product.

Examples of primary amines that may be mentioned include aromatic primary amines of the formula (C₆-C₁₀)aryl-NH₂, in which (C₆-C₁₀)aryl is, for example, phenyl, naphthyl, anthryl or phenanthryl, in which the aromatic nucleus is substituted one or more times with alkyl, alkoxy, hydroxyl or cyano.

According to another of its aspects, the invention relates to a process for acetoxylating an amine of the formula III: 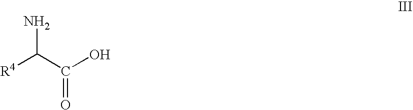
in which R⁴ represents any organic group attached to the rest of the molecule III (—CH(NH₂)—COOH) with a carbon atom, which consists in reacting the amine of the formula III with an acid of the formula R²—COOH, optionally in salified form, in which R² represents any organic group attached to the carboxylic function via a carbon atom, this reaction being performed in the presence of a polymer as defined above, so as to obtain the corresponding compound of the formula: 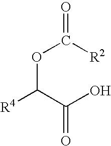
in which R² and R⁴ are as defined above.

According to one preferred embodiment of the invention, R²COOH is used in the form of an alkali metal salt and corresponds, for example, to the formula R²COO⁻,Na⁺.

R⁴ is, for example, optionally substituted alkyl; optionally substituted aryl; or optionally substituted cycloalkyl, the substituents being chosen, for example, from:
a halogen atom; cyano; hydroxyl; nitro; optionally halogenated (C₁-C₁₀)-alkyl; optionally halogenated (C₁-C₁₀)alkoxy; (C₁-C₁₀)alkylthio, which is optionally substituted by (C₆-C₁₀)arylsulphonyl, in which aryl is optionally substituted by one or more radicals G; (C₆-C₁₀)aryloxy, in which aryl is optionally substituted by one or more radicals G; (C₆-C₁₀)arylthio, in which aryl is optionally substituted by one or more radicals G; (C₁-C₁₀)alkylsulphonyl; (C₁-C₁₀)arylsulphonyl, in which aryl is optionally substituted by one or more radicals G; 5- to 7-membered heteroaryl which comprises one or more hetero atoms chosen from O, N and S and is optionally substituted by one or more radicals G and/or by (C₁-C₁₀)-alkoxycarbonyl; (C₁-C₁₀)alkoxycarbonyl; (C₁-C₁₀)alkylcarbonylamino; di(C₁-C₁₀)-alkylamino; (C₂-C₄)alkylenedioxy; (C₃-C₅)alkylene optionally substituted by oxo; (C₆-C₁₀)aryl(C₁-C₁₀)alkyl, in which aryl is optionally substituted by one or more radicals G; optionally halogenated (C₆-C₁₀)aryl; (C₁-C₁₀)alkylcarbonyl, preferably (C₁-C₆)alkylcarbonyl; (C₃-C₈)cycloalkyl(C₁-C₈)alkyl, in which cycloalkyl is itself substituted by (C₆-C₁₀)aryisulphonylamino, in which aryl is optionally halogenated; in which G is chosen from halogen; hydroxyl; optionally halogenated (C₁-C₁₄)alkoxy, preferably optionally halogenated (C₁-C₁₀)alkoxy; optionally halogenated (C₁-C₁₄)alkyl, preferably optionally halogenated (C₁-C₁₀)-alkyl; nitro; cyano; di(C₁-C₁₄)alkylamino, preferably di(C₁-C₁₀)alkylamino; (C₆-C₁₀)-aryl, which is optionally halogenated and/or optionally substituted by (C₁-C₁₄)-alkyl.

Preferably, R⁴ is optionally substituted benzyl or phenyl; R₂ is as defined above for R₄, it being understood that R₂ and R₄ are independent.

Preferably, R² is alkyl, such as CH₃.

The reaction is preferably performed at a temperature from 15 to 35° C. and better still from 20 to 25° C.

The amounts of the reagent of the formula III and of R₂COOH are usually stoichiometric.

The molar ratios of the functions A and of the compound of the formula III in the reaction medium are preferably as defined above for the nitrosation and diazotisation reactions.

The process is usually performed in the presence of a C₁-C₄ carboxylic acid, such as acetic acid.

The acetoxylation reaction described above is particularly suitable for acetoxylating a compound of the formula III in which R⁴ represents phenyl or benzyl optionally substituted by one or more substituents chosen from alkoxy, hydroxyl, cyano and alkyl.

Examples of implementation of the invention are given below.

### EXAMPLES

### Example 1

### Preparation of a Polystyrene Bearing a Function

—CH₂—NH—CO—CH(NHAc)—C(CH₃)₂—S—N═O
Step a

A solution of N-acetylpenicillamine succinimidate (1.26 g, 4.4 mmol) in DMF is added to a suspension of aminomethyl-polystyrene resin (2 g, 2.92 mmol) in DMF (10 ml). The mixture is stirred at room temperature for 24 hours and the resin is then filtered off and washed with dimethylformamide (3×20 ml), dichloromethane (3×20 ml) and MeOH (3×20 ml). After drying under vacuum, an intermediate resin containing —CH₂—NH—CO—CH(NHAc)—C(CH₃)₂—SH functions is obtained (2.36 g) in the form of a colourless resin. A negative response is obtained on a sample of resin subjected to the Kaiser calorimetric test according to the reference: E. Kaüer et al. (1970) Anal. Biochem., 34, 595. IR: 1650 (CO), 2750 (weak SH band).

Step b

The intermediate resin obtained in step a) (2.2 g, 2.57 mmol) suspended in a 10/1/0.5 dioxane/H₂O/AcOH solvent mixture (80 ml) is treated with sodium nitrite (1 g, 14.5 mmol) and the mixture is stirred at room temperature for 24 hours. The resin is then filtered off and washed with an 80/20 tetrahydrofuran (THF)/H₂O mixture (3×50 ml), THF (3×50 ml) and dichloromethane (3×50 ml). The resin is then dried under vacuum to give the expected green-coloured resin (2.2 g). IR: 1650 (CO).

### Example 2

### Preparation of a Polyethylene Glycol Bearing a Function

—CH₂—NH—CO—CH(NHAc)—C(CH₃)₂—S—N═O
Step a

The intermediate resin containing —CH₂—NH—CO—CH(NHAc)—C(CH₃)₂—SH functions is prepared according to the protocol described in step a) of Example 1. A negative response is obtained on a sample of resin subjected to the Kaiser calorimetric test.

Step b

The expected resin is prepared from the intermediate resin obtained in step a) according to the protocol described for the resin of Example 1.

### Example 3

### Preparation of a Polystyrene Bearing a Function

—CH₂—NH—CO—CH₂—C(CH₃)₂—S—N═O
Step a

A solution of 3-mercapto-3-methylbutyric acid (145 mg, 1.08 mmol) in DMF (10 ml) is added to a suspension of aminomethyl-polystyrene resin (0.5 g, 0.72 mmol) in 10 ml of dimethylformamide (DMF). 1,3-Diisopropylcarbodiimide (136 mg, 1.08 mmol) is then added dropwise. After stirring for 20 hours, the resin is filtered off and washed with DMF (3×10 ml), dichloromethane (3×10 ml) and MeOH (3×10 ml). After drying under vacuum, an intermediate resin containing —CH₂—NH—CO—CH₂—C(CH₃)₂—SH functions is obtained in the form of a colourless resin (0.54 g).

Step b

The expected resin is prepared from the intermediate resin obtained in step a) according to the protocol described for the resin of Example 1, step b).

### Example 4

### Nitrosation of 4-[(4-methoxyphenyl)amino]benzonitrile

The resin of Example 1 (50 mg, 0.048 mmol) is added to a solution of 4-[(4-methoxyphenyl)amino]benzonitrile (5 mg, 0.016 mmol) in 1.5 ml of dichloromethane (DCM). The suspension is stirred for 82 hours. The resin is then filtered off and rinsed with DCM (2×1.5 ml) and the filtrate is then evaporated under vacuum to give 4-[N-nitrosyl-(4-methoxyphenyl)amino]benzonitrile (4.9 mg, 86%). ¹H NMR (CDCl₃): 3.86 (3H, s); 6.94 (2H, m); 7.05 (2H, m); 7.54 (2H, m); 7.69 (2H, m).

### Example 5

### Acetoxylation of Phenylalanine with Sodium Acetate

The resin of Example 1 (350 mg, 0.31 mmol) is added to a solution of phenylalanine (21 mg, 0.131 mmol) in acetic acid (1 ml). A 1 M solution of sodium acetate in acetic acid (3 ml) is then added. The mixture is stirred at room temperature for 11 hours. The resin is then filtered off and rinsed with acetic acid (1 ml) and the filtrate is then evaporated under vacuum. The residue is taken up in ether (2 ml) and is then washed with water (2×2 ml). After evaporation under vacuum, 3-phenyl-2-methylcarbonyloxypropanoic acid is obtained in the form of a white powder (19.8 mg, 72%). ¹H NMR (CDCl₃); 2.01 (3H, s), 3.01-3.15 (2H, m), 5.17 (1H, dd, J=4 and 9 Hz), 7.15-7.6 (5H, m). MS ES-(m⁻¹)=207.

### Example 6

### Diazotisation of β-naphthylamine

The resin of Example 1 (100 mg, 0.110 mmol) is added to a solution of β-naphthylamine (5.3 mg, 0.037 mmol) in DCM (3 ml) in the presence of acetic acid (2.16 μl, 0.037 mmol). The mixture is stirred at room temperature for one hour. The resin is then filtered off and rinsed with DCM (2×1 ml) and the filtrate is then evaporated under vacuum without heating to give the 2-naphthalenediazonium (4 mg, 53%). ¹H NMR (CDCl₃): 6.85 (1H, d, J=9 Hz), 7.3 (1H, dt, J=1.5 and 7 Hz), 7.44-7.93 (3H, m), 8.13 (1H, dd, J=2 and 8.5 Hz), 8.25 (1H, s), 8.88 (1H, d, J=8.5 Hz).

### Examples 7 to 13

The compounds of Examples 7 to 13 below were obtained from the above secondary amines by performing the procedure described in Example 4.

| Example | | | |
|---|---|---|---|
| No. | Compound obtained | MS | ¹NMR (300 MHz) |
| 7 | 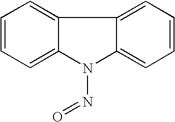 | MS ES + 167.197 | CDCl₃: 7.54-7.59 (4H, m), 7.92-8.09 (2H, m), 8.25 (1H, d), 8.56-8.59 (1H, m) |
| 8 | 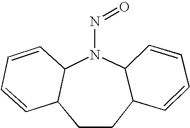 | MS ES + 225 | CDCl₃: 2.52-3.58 (4H, m); 7.05 (2H, m); 7.18-7.43 (4H, m); 7.61 (2H, m) |
| 9 | 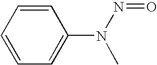 | MS ES + 137 | CDCl₃: 3.5 (3H, s), 7.35-7.70 (5H, m) |
| 10 | 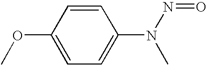 | MS ES + 137.167 | CDCl₃; 3.44 (3H, s), 3.85 (3H, s), 7.98 (2H, d, J=9 Hz), 7.43 (2H, d, J=9 Hz) |
| 11 | 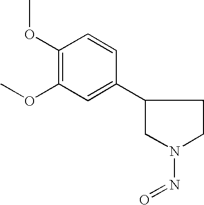 | MS ES + 237 | Mixture of cis/trans isomers CDCl₃: 1.90-2.57 (2H, m); 3.02-3.69 (3H, m); 3.69-4.01 (1 H, m + 6H, s); 6.57-7.01 (3H, m) |
| 12 | 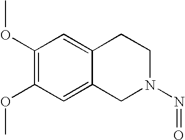 | MS ES + 223 | Mixture of cis/trans isomers CDCl₃: 3.02 (2H, m); 3.86 6H, 2s); 4.51 (2H, m); 4.76 (2H, m); 6.51-6.88 (2H, m) |
| 13 | 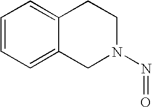 | MS ES + 163 | Mixture of cis/trans isomers CDCl₃: 2.96 (2H, t, J=6.5 Hz), 3.10 (2H, t, J=6.5 Hz), 3.88 (2H, t, J=6.5 Hz), 4.54 (2H, t, J=6.5 Hz), 4.83 (2H, s), 5.39 (2H, s), 7.14-7.28 (4H, m) |

### Examples 14 and 15

The compounds of Examples 14 and 15 were obtained from the corresponding starting amines by performing the procedure described in example 5.

| Example | | Starting | |
|---|---|---|---|
| No. | Compound obtained | material | ¹H NMR (300 MHz) |
| 14 | 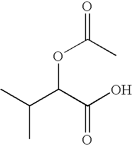 | 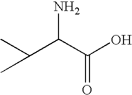 | CDCl₃: 0.87 (6H, broad s), 2.02 (4H, m), 4.54 (1H, m) |
| 15 | 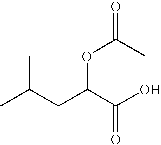 | 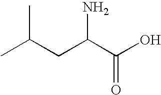 | CDCl₃: 0.9-1 (6H, 2d), 1.65-1.88 (3H, m), 2.15 (3H, s), 5.08(1 H, m) |

1. Polymer bearing at least one function of the formula A: 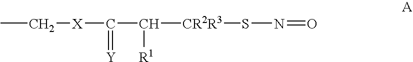
in which:
X represents O, S or NT, in which T represents H or a saturated aliphatic hydrocarbon-based group; Y represents O or S;
R¹ represents H; a saturated aliphatic hydrocarbon-based group, which is optionally substituted and/or optionally interrupted by one or more O or S atoms; a group —(O)ₚ—Ar, in which p represents the integer 0 or 1 and Ar represents an optionally substituted saturated and/or aromatic carbocyclic group or an optionally substituted saturated and/or aromatic heterocyclic group; a group Hyd-CO—O— or Hyd-CO—NH—, in which Hyd represents an optionally substituted saturated aliphatic hydrocarbon-based group; an optionally substituted aromatic group; or an optionally substituted aromatic heterocyclic group;
R² and R³ represent, independently of each other, a hydrogen atom; an optionally substituted saturated aliphatic hydrocarbon-based group; a saturated and/or aromatic carbocyclic group.

2. Polymer according to claim 1, of styrene or polyethylene glycol type.

3. Polymer according to claim 1, characterised in that X represents N.

4. Polymer according to claim 1, characterised in that R² and R³ independently represent H, (C₁-C₈)alkyl; (C₆-C₁₀)aryl; and R¹ represents H; (C₁-C₈)alkyl; (C₆-C₁₀)aryl; (C₆-C₁₀)aryloxy; heteroaryl which comprises one or more hetero atoms chosen from O, S and N and consists of one or more 5- to 8-membered monocycles; heteroaryloxy, in which heteroaryl is as defined above; (C₁-C₈)alkylcarbonyloxy; (C₁-C₈)alkylcarbonylamino; (C₆-C₁₀)arylcarbonyloxy; (C₆-C₁₀)arylcarbonylamino; heteroarylcarbonyloxy; or hetero-arylcarbonyl-amino, in which heteroaryl is as defined above.

5. Polymer according to claim 4, characterised in that R² and R³ independently represent (C₁-C₄)alkyl; and R¹ represents (C₁-C₄)alkyl-carbonylamino.

6. Polymer according to claim 1, characterised in that the function A is:
—CH₂—O—CO—CH(NH—W)—C(CH₃)₂—S—N═O A1
in which W represents alkanoyl (i.e. alkylcarbonyl), such as acetyl.

7. Polymer according to claim 6, characterised in that it is of polystyrene or polyethylene glycol type.

8. Process for the preparation of a polymer according to claim 1, characterised in that it comprises the steps consisting in:
a) reacting a precursor polymer P bearing at least one —CH₂—XH function with the required amount of a reagent of the formula I: 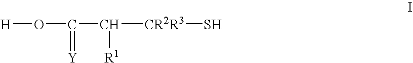
in which the HO—C(═Y)— function is optionally in activated form and Y, R¹, R² and R³ are as defined in claim 1, such that each —CH₂—XH function of the polymer P reacts with a molecule of the reagent of the formula I, and then
b) treating the resulting polymer bearing at least one function B: 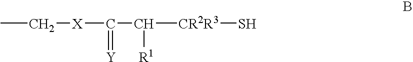
with a nitrosating agent, so as to convert each of the functions B into functions C:

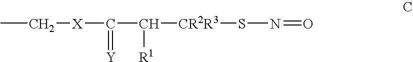

9. Process according to claim 8, characterised in that the nitrosating agent is chosen from an alkali metal nitrite; an alkyl nitrite; or NO⁺BF₄⁻.

10. Process for nitrosating secondary amines, which consists in reacting a secondary amine with a polymer according to claim 1, so as to obtain the corresponding nitroso derivative.

11. Process according to claim 10, characterised in that the molar ratio of the functions of the formula A of the polymer to the secondary amine functions ranges between 2 and 10 and preferably between 2 and 5.

12. Process according to claim 10, characterised in that it is performed at a temperature of between 15 and 35° C.

13. Process according to claim 10, characterised in that the secondary amine is chosen from diphenylamines optionally substituted by one or more substituents of alkyl, alkoxy, cyano or hydroxyl type; an amine of the formula: 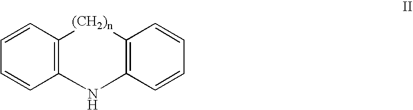
in which n is an integer equal to 0, 1, 2 or 3 and the phenyl nuclei are optionally independently substituted by one or more substituents chosen from alkyl, cyano, alkoxy and hydroxyl; a phenylalkylamine optionally substituted by one or more substituents chosen from alkyl, cyano, alkoxy and hydroxyl; a benzopyrrolidine or a benzopiperidine optionally substituted by one or more substituents chosen from hydroxyl, alkyl, cyano and alkoxy; a benzylpyrrolidine optionally substituted by one or more substituents chosen from hydroxyl, cyano, alkyl and alkoxy; or a benzylpiperidine optionally substituted by one or mole substituents chosen from alkyl, cyano, alkoxy and hydroxyl.

14. Process for diazotising primary amines, which consists in reacting a primary amine with a polymer according to claim 1, so as to obtain the corresponding diazonium derivative.

15. Process according to claim 14, characterised in that the amine is an aromatic amine optionally substituted by one or more substituents chosen from hydroxyl, cyano, alkoxy and alkyl.

16. Process for acetoxylating an amine of the formula III: 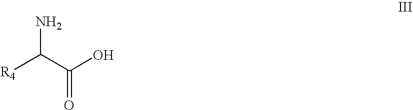
in which R⁴ represents any organic group attached to the rest of the molecule III (—CH(NH₂)—COOH) via a carbon atom, which consists in reacting the amine of the formula III with an acid of the formula R²—COOH optionally in salified form, in which R² represents any organic group attached to the carboxylic function via a carbon atom, in the presence of a polymer according to claim 1, so as to obtain the corresponding compound of the formula: 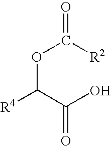
in which R² and R⁴ are as defined above.

17. Process according to claim 16, characterised in that R⁴ represents phenyl or benzyl optionally substituted by one or more alkoxy, hydroxyl, cyano or alkyl.
